(19) United States
(12) Patent Application Publication
(10) Pub. No.: US 2022/0211327 A1
(43) Pub. Date: Jul. 7, 2022

(54) MEASUREMENT METHOD AND APPARATUS FOR PERIOD INFORMATION OF BIOLOGICAL SIGNAL, AND ELECTRONIC DEVICE

(71) Applicant: BOE TECHNOLOGY GROUP CO., LTD., Beijing (CN)

(72) Inventor: Chunshan ZU, Beijing (CN)

(73) Assignee: BOE TECHNOLOGY GROUP CO., LTD., Beijing (CN)

(21) Appl. No.: 17/438,597

(22) PCT Filed: Feb. 2, 2021
(86) PCT No.: PCT/CN2021/074907

§ 371 Date: Sep. 13, 2021

(30) Foreign Application Priority Data
Mar. 31, 2020 (CN) 202010244231.2

(51) Int. Cl. A61B 5/00; A61B 5/08; A61B 5/0205; A61B 5/318; A61B 5/369; A61B 5/389; G06K 9/00; G06N 3/04

(52) U.S. Cl. CPC A61B 5/7267; A61B 5/0816; A61B 5/0205; A61B 5/318; A61B 5/369; G06N 3/0454; A61B 5/7203; A61B 5/7235; G06K 9/00523; G06K 9/00536; A61B 5/389

(57) ABSTRACT

A measurement method and an apparatus for period information of a biological signal, an electronic device and a computer-readable storage medium are provided. The measurement method includes: acquiring a feature tensor of a biological signal, classifying the feature tensor by using each of a plurality of binary classifiers to obtain a plurality of classification results, classification parameters of each of the plurality of binary classifiers being different, and determining a first measurement value of the period information of the biological signal based on the plurality of classification results.

The present application claims the priority of the Chinese patent application No. 2020 1 024423 1.2 filed on Mar. 31, 2020, and the entire disclosure of the above Chinese patent application is incorporated herein by reference as part of the disclosure of the present application.

### TECHNICAL FIELD

Embodiments of the present disclosure relate to a measurement method and an apparatus for period information of a biological signal, an electronic device and a computer storage medium.

### BACKGROUND

With the increase of people's demand for health and medical treatment, research on the integration of information technology (IT) and medicine is being carried out. In particular, it is no longer limited that performing monitoring of the health status of a human body in a fixed place such as a hospital, but the health status of a user can be monitored anywhere (such as at home or in the office) and at any time. The user's health status can be represented by the user's biological signal. The biological signal may comprise an electrocardiograph signal, a respiratory signal, an electroencephalogram signal, a pulse signal (such as a photoplethysmography signal), an electromyography signal, etc. By analyzing information of the biological signal, the physical and mental state of the user in daily life can be monitored.

### SUMMARY

According to at least one embodiment of the present disclosure, a measurement method for period information of a biological signal is provided. The measurement method comprises: acquiring a feature tensor of the biological signal; classifying the feature tensor by using each of a plurality of binary classifiers to obtain a plurality of classification results, and classification parameters of each of the plurality of binary classifiers being different; and determining a first measurement value of the period information of the biological signal based on the plurality of classification results.

For example, in some embodiments, before classifying the feature tensor by using each of the plurality of binary classifiers, the measurement method further comprises: determining a range of the period information of the biological signal; and determining an amount of the plurality of binary classifiers and the classification parameters of each of the plurality of binary classifiers based on the range.

For example, in some embodiments, classifying the feature tensor by using each of the plurality of binary classifiers to obtain the plurality of classification results comprises: converting the feature tensor into a feature matrix; and classifying the feature matrix by using each of the plurality of binary classifiers to obtain the plurality of classification results.

For example, in some embodiments, each of the plurality of binary classifiers is implemented as a convolutional neural network.

For example, in some embodiments, acquiring the feature tensor of the biological signal comprises extracting features of the biological signal through the convolution neural network so as to obtain the feature tensor.

For example, in some embodiments, the measurement method further comprises: dividing the feature tensor into a plurality of segments; estimating a feature point in each of the plurality of segments to obtain a plurality of estimation results, and the feature point being associated with the period information of the biological signal; determining a second measurement value of the period information of the biological signal based on the plurality of estimation results; and obtaining a final measurement value of the period information of the biological signal based on the first measurement value and the second measurement value.

For example, in some embodiments, the measurement method further comprises: dividing the feature tensor into a plurality of segments; estimating a feature point in each of the plurality of segments to obtain a plurality of estimation results, and the feature point being associated with the period information of the biological signal; and determining a second measurement value of the period information of the biological signal based on the plurality of estimation results. The biological signal and the second measurement value further serve as training data for training of each of the plurality of binary classifiers.

For example, in some embodiments, dividing the feature tensor into the plurality of segments comprises: converting the feature tensor into a feature matrix; and dividing the feature matrix into the plurality of segments.

For example, in some embodiments, a convolutional neural network is used to estimate the feature point in each of the plurality of segments.

For example, in some embodiments, the plurality of estimation results are further used for training of the convolutional neural network.

For example, in some embodiments, the measurement method further comprises: training each of the plurality of binary classifiers. The training comprises: determining a training data set for the plurality of binary classifiers; and training each of the plurality of binary classifiers by using the training data set.

For example, in some embodiments, training each of the plurality of binary classifiers by using the training data set comprises: for each of the plurality of binary classifiers, dividing the training data set into a first subset and a second subset, and training a corresponding binary classifier by using the first subset and the second subset.

For example, in some embodiments, a type of the biological signal comprises at least one selected from a group consisting of an electrocardiograph signal, a respiratory signal, a pulse signal, an electroencephalogram signal, and an electromyography signal.

According to at least one embodiment of the present disclosure, a measurement apparatus for period information of a biological signal is provided. The measurement apparatus comprises: a feature acquisition module which is configured to acquire a feature tensor of the biological signal; a classification module which is configured to classify the feature tensor by using each of a plurality of binary classifiers to obtain a plurality of classification results, and classification parameters of each of the plurality of binary classifiers being different; and a period information determination module which is configured to determine a first measurement value of the period information of the biological signal based on the plurality of classification results.

For example, in some embodiments, the classification module is used to determine a range of the period information of the biological signal, and determine an amount of the plurality of binary classifiers and classification parameters of each of the plurality of binary classifiers based on the range.

For example, in some embodiments, the classification module is used to convert a feature tensor into a feature matrix, and classify the feature matrix by using each of the plurality of binary classifiers to obtain the plurality of classification results.

For example, in some embodiments, the measurement apparatus further comprises a training module, which is used to determine a training data set and train each of the plurality of binary classifiers by using the training data set.

For example, in some embodiments, the training module is used to divide the training data set into a first subset and a second subset for each of the plurality of binary classifiers, and train a corresponding binary classifier by using the first subset and the second subset.

For example, in some embodiments, the measurement apparatus further comprises a segmentation measurement module. The segmentation measurement module comprises: a segmentation sub-module which is configured to divide the feature tensor into a plurality of segments; an estimation sub-module which is configured to estimate a feature point in each of the plurality of segments to obtain a plurality of estimation results, and the feature point being associated with the period information of the biological signal; and a second period information determination sub-module which is configured to determine a second measurement value of the period information of the biological signal based on the plurality of estimation results. The biological signal and the second measurement value are further used for training of each of the plurality of binary classifiers as training data.

For example, in some embodiments, the measurement apparatus further comprises a segmentation measurement module, which comprises: a segmentation sub-module which is configured to divide a feature tensor into a plurality of segments; an estimation sub-module which is configured to estimate the feature point in each of the plurality of segments to obtain a plurality of estimation results, and the feature point being associated with the period information of the biological signal; and a second period information determination sub-module which is configured to determine the second measurement value of the period information of the biological signal based on the plurality of estimation results. The plurality of estimation results are further used for the training of the estimation sub-module.

For example, in some embodiments, the segmentation sub-module is used to convert the feature tensor into a feature matrix and divide the feature matrix into the plurality of segments.

For example, in some embodiments, a type of the biological signal comprises at least one selected from the group consisting of an electrocardiograph signal, a respiratory signal, a pulse signal, an electroencephalogram signal, and an electromyography signal.

According to at least one embodiment of the present disclosure, an electronic device is provided. The electronic device comprises: at least one processor; and a memory which is configured to store at least one computer program. In the case where the at least one computer program is executed by the at least one processor, the at least one processor executes one or more of the steps of the measurement method described in any one of the above embodiments.

For example, in some embodiments, the electronic device further comprises one or more sensors for acquiring the biological signal to be measured.

According to at least one embodiment of the present disclosure, a computer-readable storage medium, on which at least one computer program is stored, is provided. In the case where the at least one computer program is executed by at least one processor, the at least one computer program executes one or more of the steps of the measurement method described in any one of the above embodiments.

### BRIEF DESCRIPTION OF THE DRAWINGS

In order to illustrate the technical solutions of the embodiments of the present disclosure more clearly, the following will briefly introduce the attached drawings of the embodiments. Obviously, the attached drawings in the following description merely relate to some embodiments of the present disclosure and are not a limitation of the present disclosure.

FIG. 1 illustrates a block diagram of a measurement apparatus for period information of a biological signal according to some embodiments of the present disclosure;

FIG. 2 illustrates a block diagram of a classification module according to some embodiments of the present disclosure;

FIG. 3 illustrates a block diagram of a measurement apparatus for period information of a biological signal according to some embodiments of the present disclosure;

FIG. 4A illustrates a schematic diagram of a plurality of segments obtained by dividing a feature matrix according to some embodiments of the present disclosure;

FIG. 4B illustrates a schematic diagram of estimating segments by an estimation model comprised in an estimation sub-module according to some embodiments of the present disclosure;

FIG. 5 illustrates a flowchart of a measurement method for period information of a biological signal according to some embodiments of the present disclosure;

FIG. 6 illustrates a flowchart of a measurement method for period information of a biological signal according to some embodiments of the present disclosure; and

FIG. 7 illustrates a block diagram of an electronic device according to some embodiments of the present disclosure.

### DETAILED DESCRIPTION

In order to make the purpose, technical solutions and advantages of the embodiments of the present disclosure clearer, the technical solutions in the embodiments of the present disclosure will be described clearly and completely in combination with the attached drawings. Obviously, the described embodiments are only a part of the embodiments of the present disclosure, not all of them. Based on the described embodiments of the present disclosure, all other embodiments obtained by the person of ordinary skill in the art without making creative work shall belong to the protection scope of the present disclosure.

The terms used herein to describe the embodiments of the present disclosure are not intended to restrict and/or limit the scope of the present disclosure.

For example, unless otherwise defined, the technical or scientific terms used in the present disclosure shall have usual meanings understood by those with ordinary skills in the field to which this disclosure belongs.

It should be understood that, the words “first”, “second” and similar words used in the present disclosure do not mean any order, quantity, or importance, but are only used to distinguish different components. Unless the context clearly indicates otherwise, similar words such as “an”, “a” or “the” in singular form do not mean a quantity limit, but instead mean that there is at least one.

It will be further understood that, similar words such as “including” or “comprising” mean that the elements or items appearing before the word cover the elements or items listed after the word and their equivalents, but do not exclude other elements or items. Similar words such as “connecting” or “connected” are not limited to physical or mechanical connections, but can comprise electrical connections, whether direct or indirect. “Up”, “Down”, “Left”, “Right”, etc. are only used to indicate the relative position relationship, and in the case that the absolute position of the described object changes, the relative position relationship may also change accordingly.

A biological signal may comprise an electrocardiograph signal, a respiratory signal, an electroencephalogram signal, a pulse signal (such as a photoplethysmography signal), an electromyography signal, etc. By analyzing the information of the biological signal, the physical and mental state of the user in daily life can be monitored.

In some cases, the user's health information can be estimated by analyzing period information of the biological signal. The period information of the biological signal may comprise one or more of heart rate, respiratory rate, pulse rate, blink rate, etc.

The period information of the biological signal can be measured by regression analysis or curve fitting. However, this method is easily affected by interference, thereby resulting in low accuracy of measurement results.

In order to solve at least the above problems, the embodiments of the present disclosure provide a method and an apparatus for measuring period information of a biological signal, an electronic device and a computer-readable storage medium.

Hereinafter, the embodiments of the present disclosure will be described in detail with reference to the attached drawings. It should be noted that the same reference numerals in different drawings will be used to refer to the same elements that have been described.

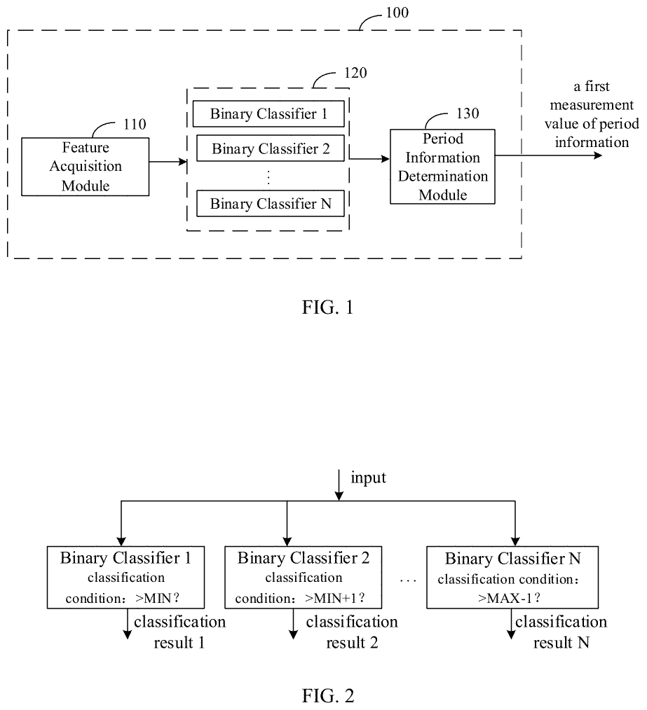

FIG. 1 illustrates a block diagram of a measurement apparatus for period information of a biological signal according to some embodiments of the present disclosure.

Referring to FIG. 1, the measurement apparatus 100 may comprise a feature acquisition module 110, a classification module 120, and a period information determination module 130. The feature acquisition module 110 is used to acquire a feature tensor of a biological signal. The classification module 120 may comprise a plurality of binary classifiers (a binary classifier 1, a binary classifier 2, . . . , a binary classifier N). The period information determination module 130 is used to determine a first measurement value of the period information of the biological signal based on the plurality of classification results.

In the embodiments of the present disclosure, the biological signal comprises at least one selected from the group consisting of an electrocardiograph signal, a respiratory signal, a pulse signal, an electroencephalogram signal, and an electromyography signal. The period information of the biological signal may comprise one or more of heart rate, respiratory rate, pulse rate, blink rate, etc.

In the embodiments of the present disclosure, the feature tensor may comprise one or more matrices for characterizing various features of the biological signal. In this case, elements in the feature tensor are related to the features of the biological signal.

In an example, features related to the period information of the biological signal can be extracted to generate a third-order feature tensor. For example, in the case that the biological signal is a multi-lead biological signal (for example, a multi-lead electrocardiograph signal), a third-order feature tensor can be generated. In this case, the feature tensor characterizes the timing information related to the period information of the biological signal and the spatial information indicating the timing correlation between each lead biological signal in the biological signal.

In another example, the features related to the period information of the biological signal can be extracted to generate a second-order feature tensor, that is, a feature matrix. For example, in the case that the biological signal is a single-lead biological signal (for example, a single-lead electrocardiograph signal), a second-order feature tensor (i.e., a feature matrix) can be generated. In this case, the feature tensor characterizes the timing information related to the period information of the biological signal.

In the case that the biological signal is an electrocardiograph signal, examples of the features which are related to the period information of the biological signal may comprise one or more of an R-wave peak (for example, an R-wave vertex), a T wave peak (for example, a T wave vertex), a QRS start point, or a QRS end point.

In some embodiments, the feature acquisition module 110 can be used to extract features of a biological signal to obtain a feature tensor.

In some embodiments, the feature acquisition module 110 can receive a feature tensor of a biological signal from another device.

In some embodiments, the feature acquisition module 110 may comprise a first neural network model 110. The first neural network model 110 may comprise a feedforward neural network, a recurrent neural network (RNN), a convolutional neural network (CNN), or other forms of neural networks. The type of the first neural network model may comprise a depth neural network.

Although embodiments that can be implemented as various neural networks by the feature acquisition module 110 have been described, the present disclosure is not limited thereto. For example, the feature acquisition module 110 can be implemented as any machine learning model that can acquire the feature tensor.

In some embodiments, each of the binary classifier 1, the binary classifier 2, . . . , the binary classifier N can be implemented as a neural network. For example, the neural network for the classification module may comprise a feedforward neural network, a recurrent neural network (RNN), a convolutional neural network (CNN), or other forms of neural networks. The type of the neural network which is used for the classification module may comprise the depth neural network.

In some embodiments, referring to FIG. 2, the inputs of the plurality of binary classifiers in the classification module 120 are the same. That is, the plurality of binary classifiers classify the same input. For example, the input of the plurality of binary classifiers can be the feature tensor of the biological signal which is acquired by the feature acquisition module 110. In an example, the input of the plurality of binary classifiers can be the feature vector which is obtained by converting the feature tensor.

In some embodiments of the present disclosure, the feature tensor can be converted into a feature matrix by a matricization method of the tensor. The matricization method of the tensor that can be applied to some embodiments of the present disclosure is described below by some specific examples. For ease of description, it is assumed that the tensor to be matrixed is Γ:

$$Γ = \{ [ \begin{matrix}{a}_{11} & {a}_{12} & {a}_{13} \\ {a}_{21} & {a}_{22} & {a}_{23}\end{matrix} ] ; [ \begin{matrix}{b}_{11} & {b}_{12} & {b}_{13} \\ {b}_{21} & {b}_{22} & {b}_{23}\end{matrix} ] ; [ \begin{matrix}{c}_{11} & {c}_{12} & {c}_{13} \\ {c}_{21} & {c}_{22} & {c}_{23}\end{matrix} ] \} ,$$

the order of the tensor Γ is 3 and the size is 2×3×3. Because the tensor can be regarded as being composed of a plurality of matrices, the tensor Γ can also be expressed as Γ={A; B; C}, and matrices A, B and C are respectively expressed as:

$$A = [ \begin{matrix}{a}_{11} & {a}_{12} & {a}_{13} \\ {a}_{21} & {a}_{22} & {a}_{23}\end{matrix} ] , \\ B = [ \begin{matrix}{b}_{11} & {b}_{12} & {b}_{13} \\ {b}_{21} & {b}_{22} & {b}_{23}\end{matrix} ] , \\ C = [ \begin{matrix}{c}_{11} & {c}_{12} & {c}_{13} \\ {c}_{21} & {c}_{22} & {c}_{23}\end{matrix} ] .$$

In an example, a corresponding matrix can be obtained by extending the tensor in a certain direction (for example, in the column direction). For example, for the tensor Γ, by splicing in the column direction, a matrix T which corresponds to the tensor Γ can be obtained:

$$T = [ \begin{matrix}{a}_{11} & {a}_{12} & {a}_{13} \\ {a}_{21} & {a}_{22} & {a}_{23} \\ {b}_{11} & {b}_{12} & {b}_{13} \\ {b}_{21} & {b}_{22} & {b}_{23} \\ {c}_{11} & {c}_{12} & {c}_{13} \\ {c}_{21} & {c}_{22} & {c}_{23}\end{matrix} ] .$$

In another example, data processing (for example, taking the mean value) can be performed on the element of the corresponding position of the plurality of matrices which constitute the feature tensor, so as to convert the feature tensor into a feature matrix. For example, by performing data processing on the elements at the same position of matrices A, B and C, a matrix Q which corresponds to the tensor Γ can be obtained:

$$Q = [ \begin{matrix}{q}_{11} & {q}_{12} & {q}_{13} \\ {q}_{21} & {q}_{22} & {q}_{23}\end{matrix} ] .$$

In the above formula, each element in the matrix Q can be a value which is obtained by performing data processing on the elements at the same position of matrices A, B and C. For example, q₁₁ can be a value which is obtained by performing data processing (for example, taking the mean value) on a₁₁, b₁₁, and c₁₁, q₁₂ can be a value which is obtained by performing data processing (for example, taking the mean value) on a₁₂, b₁₂, and c₁₂, and q₂₃ can be a value which is obtained by performing data processing (for example, taking the mean value) on a₂₃, b₂₃, and c₂₃. Other elements (q₁₃, q₂₁, q₂₂) in matrix Q can be values which are obtained in a similar manner.

It should be noted that the order and size of the tensor described above are only exemplary. In addition, although the matricization method of the tensor that can be applied to some embodiments of the present disclosure is described above, the embodiments of the present disclosure are not limited thereto. For example, various methods can be adopted to convert a feature tensor into a feature matrix.

In some embodiments, the amount of the plurality of binary classifiers comprised in the classification module 120 can be determined based on the range of the period information. In an example, if the range of the period information is expressed as MIN˜MAX, and MIN and Max are non-negative integers, the total amount of categories K can be expressed as: K=MAX−MIN+1, and category 1 to category K can correspond to MIN, MIN+1, . . . , Max, respectively. In this case, the amount (i.e., N) of the plurality of binary classifiers comprised in the classification module 120 can be determined as K−1. Moreover, the desired classification thresholds of the binary classifier 1, the binary classifier 2, . . . , the binary classifier N can be determined as MIN, MIN+1, . . . , MAX−1, respectively. That is, the classification threshold of the binary classifier 1 can be MIN, and the classification threshold of the binary classifier 2 can be MIN+1. Similarly, the classification threshold of the binary classifier N can be MAX−1. In the embodiments of the present disclosure, by training each of the plurality of classifiers, the corresponding classifier can have corresponding model parameters (referred to as classification parameters in the present disclosure) and can have the ability to identify whether inputs meet the classification condition related to the classification threshold. For example, after training, the binary classifier 1 can identify whether an input is greater than the classification threshold MIN, the binary classifier 2 can identify whether an input is greater than the classification threshold MIN+1, and the binary classifier N can identify whether an input is greater than the classification threshold MAX−1.

For example, each of the plurality of binary classifiers comprised in the classification module 120 can perform classification based on its classification threshold and obtain classification results. In an example, in the case that an input of the binary classifier meets the corresponding classification condition, the classification result can be “1”, and in the case that an input of the binary classifier does not meet the corresponding classification condition, the classification result can be “0”. In another example, in the case that an input of the binary classifier meets the corresponding classification condition, the classification result can be “1”, and in the case that an input of the binary classifier does not meet the corresponding classification condition, the classification result can be “−1”.

In some embodiments, the period information determination module 130 can determine a first measurement value of the period information of the biological signal based on equation (1).

$$p ( {x}_{i} ) = MIN + {\sum }_{n = 1}^{N} [ {f}_{n} ( {x}_{i} ) > 0 ] \qquad [ equation ( 1 ) ]$$

In equation (1), p(xᵢ) represents the first measurement value of the period information of the biological signal xᵢ, MIN can represent the minimum value within the range of the period information, n represents the number of the binary classifier, xᵢ is the input of each of the N binary classifiers (the binary classifier 1, the binary classifier 2, . . . , the binary classifier N), ƒₙ(xᵢ) is the output of the corresponding binary classifier, and [.] is an indicative function. For example, [ƒₙ(xᵢ)>0] can represent in the case that the expression “ƒₙ(xᵢ)>⁰” is true, 1 is returned, otherwise 0 is returned. In addition, N can be determined as MAX-MIN, and MAX can represent the maximum value within the range of the period information. Thus, based on the classification result of each of the plurality of binary classifiers, the first measurement value of the period information of the biological signal can be obtained.

In some embodiments, the measurement apparatus 100 can further comprise a training module (not illustrated). The training module can be used to train the plurality of binary classifiers comprised in the classification module 120. In addition, the training module can further be used to train the feature acquisition module 110.

For example, the measurement method further comprises training the plurality of binary classifiers before using the plurality of binary classifiers in the classification module 120. Some examples of the training method are described below.

In some examples, assuming y_{f} is the feature vector representing the j-th training sample (biological signal), tⱼ∈{MIN, MIN+1, . . . . . . , MAX} is the corresponding sample category label, which indicates the category of the corresponding training sample. For each binary classifier, the entire training data set D is divided into two subsets, as shown in equation (2) and equation (3).

Dₙ⁺={(yⱼ,+1)|tⱼ>(MIN+n−1)} [equation (2)]

Dₙ⁻={yⱼ−1)|tⱼ≤(MIN+n−1)} [equation (3)]

In equation (2) and equation (3), n represents the number of the binary classifier, n is an integer greater than or equal to 1 and less than or equal to N, Dₙ⁺ and Dₙ⁻ represent two subsets of the binary classifier n (the first subset Dₙ⁺ and the second subset Dₙ⁻),“+1” and “−1” represent binary classification labels. The binary classification labels can represent the classification results obtained by classifying them by using the binary classifiers. For the binary classifier n, Dₙ⁺ comprises a training sample whose sample category label is greater than (MIN+n−1), and the binary classification label of the training sample is determined as “+1”, Dₙ⁻ comprises a training sample whose sample category label is less than or equal to (MIN+n−1), and the binary classification label of the training sample is determined as “−1”.

For each binary classifier, two subsets obtained by dividing the training data set D are used for training to obtain the parameters of each binary classifier or optimize each binary classifier. Therefore, for each binary classifier, all training samples in the training data set D are used for training, thereby it is not easy to be affected by local interference.

In some embodiments, training data can be used to jointly train one or more of the feature acquisition module 110, the classification module 120, and the period information determination module 130 in the measurement apparatus 100. That is, the feature acquisition module 110, the classification module 120 and the period information determination module 130 in the measurement apparatus 100 can form an end-to-end learning model.

In some embodiments of the present disclosure, the plurality of binary classifiers comprised in the classification module 120 are trained in a multi-task manner based on different divisions. Because each binary classifier uses all the training samples in the training data set, it is not easy to be affected by local interference and is not easy to overfit. For example, overfitting refers to the phenomenon that a model (for example, a binary classifier) performs well on the training data set and poorly on the test data set.

The period information of the biological signal has significant incremental characteristic. For example, heart rate increases monotonically with the shortening of the heartbeat cycle. Moreover, the biological signal itself is easily disturbed by random noise thereby resulting in randomness of its period information. In the embodiments of the present disclosure, the influence of random noise on measurement results can be reduced by setting a plurality of binary classifiers for classification by using the incremental characteristic of the period information of the biological signal.

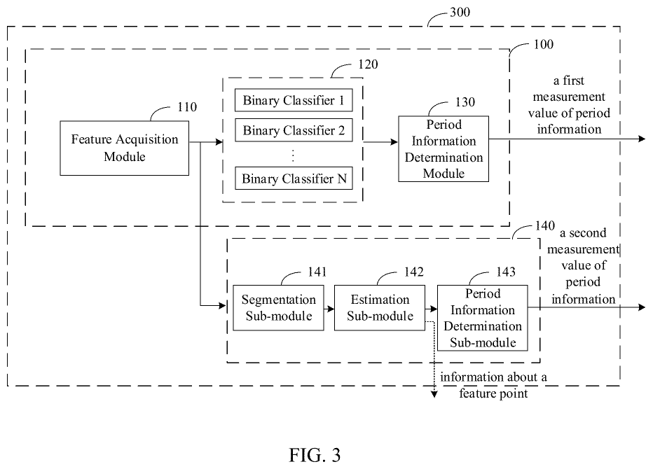

FIG. 3 illustrates a block diagram of the measurement apparatus 300 for period information of a biological signal according to some embodiments of the present disclosure.

Referring to FIG. 3, in addition to the feature acquisition module 110, the classification module 120 and the period information determination module 130 illustrated in FIG. 1, the apparatus 300 further comprises a segmentation measurement module 140.

For the configuration of the feature acquisition module 110, the classification module 120 and the period information determination module 130, reference can be made to the above description, and further descriptions thereof are omitted here.

Continuing to refer to FIG. 3, the segmentation measurement module 140 may comprise a segmentation sub-module 141, an estimation sub-module 142, and a period information determination sub-module 143.

The segmentation sub-module 141 is used to divide a feature tensor into a plurality of segments.

For example, the segmentation sub-module 141 can be used to convert the feature tensor into a feature matrix and divide the feature matrix into a plurality of segments based on column vectors of the feature matrix, and each of the plurality of segments corresponds to a column vector in the feature matrix.

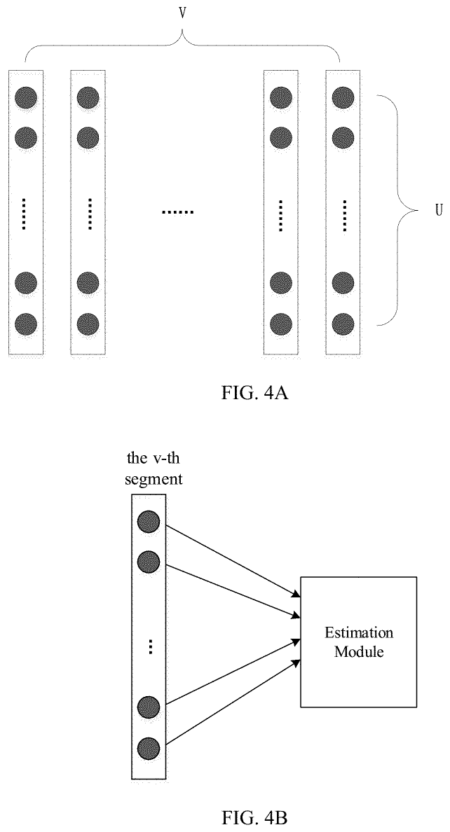

In some embodiments of the present disclosure, the feature tensor can be converted into a feature matrix by a matricization method of the tensor. For the method of converting the feature tensor into the feature matrix, reference can be made to various examples described above. FIG. 4A illustrates a schematic diagram of a plurality of segments obtained by dividing the feature matrix. Referring to FIG. 4A, for the feature matrix with a size of U×V, the feature matrix can be divided into V column vectors as V segments, and U and V are both positive integers.

The estimation sub-module 142 is used to estimate a feature point in each of the plurality of segments to obtain a plurality of estimation results, and the feature point is associated with the period information of the biological signal.

In some embodiments, in the case that the biological signal is an electrocardiograph signal, the feature point may comprise an R-wave peak (for example, an R-wave vertex). In this case, the period information of the biological signal can be measured by the amount of feature points of the same kind appearing in a certain period of time. For example, heart rate can be determined by the amount of R-wave vertices appearing in an electrocardiograph signal for a predetermined time period (for example, one minute).

In some embodiments, in the case that the feature matrix is divided, it is ensured that the amount of feature points in each of the plurality of segments does not exceed one. In this case, the period information of the biological signal can be obtained by estimating the amount of segments with feature points in the plurality of segments by the estimation sub-module 142.

In some embodiments, the estimation sub-module 142 may comprise a plurality of estimation models. For example, the amount of the plurality of estimation models is the same as the amount of segments which are obtained by division. Each estimation model corresponds to a segment and is used to estimate the corresponding segment so as to obtain a corresponding estimation result. Each estimation model can be used to estimate whether the corresponding segment has a feature point and output a corresponding estimation result. Referring to FIG. 4B, for the v-th segment (i.e., the v-th column vector), v is an integer greater than or equal to 1 and less than or equal to V, and a corresponding estimation model can be used to estimate the amount of feature points in this segment.

In some embodiments, the estimation sub-module 142 may comprise a single estimation model through which the plurality of segments divided by the segmentation sub-module are respectively estimated to obtain a plurality of estimation results.

In some embodiments, the estimation sub-module 142 or the (plurality of) estimation model(s) comprised therein can be implemented as a neural network. For example, the neural network for the estimation sub-module 142 may comprise a feedforward neural network, a recurrent neural network (RNN), a convolutional neural network (CNN), or other forms of neural network. The type of the neural network which is used for the classification module can comprise a depth neural network.

The period information determination sub-module 143 is used to determine a second measurement value of the period information of the biological signal based on the plurality of estimation results obtained by the estimation sub-module 142.

In some embodiments, the amount of feature points in each of the plurality of segments divided by the segment sub-module 141 does not exceed one. In this case, the second measurement value of the period information of the biological signal can be determined based on equation (4).

$$q ( {z}_{l} ) = {\sum }_{v = 1}^{V} [ {g}_{v} ( {z}_{l} ) > 0 ] \qquad [ equation ( 4 ) ]$$

In equation (4), q(zᵢ) represents the second measurement value of the period information of the biological signal zₒ v represents the number of the segment, gᵥ(zᵢ) represents the estimation result output by the estimation model corresponding to the v-th segment, V represents the amount of the plurality of segments, and [.] is an indicative function. For example, [gᵥ(zᵢ)>0] can represent in the case that the expression “gᵥ(zᵢ)>0” is true, 1 is returned, otherwise 0 is returned.

In some embodiments, one of the first measurement value and the second measurement value can be taken as the final measurement value, and the final measurement value is taken as the period information of the biological signal which is obtained by measurement.

In some embodiments, a final measurement value of the period information of the biological signal is obtained based on the first measurement value and the second measurement value, and the final measurement value is taken as the period information of the biological signal which is obtained by measurement. For example, the first measurement value and the second measurement value can be weighted and averaged to obtain the final measurement value.

In some embodiments, the biological signal and the second measurement value of the period information corresponding to this biological signal can be taken as a training sample. For example, the training sample can be used for training of the plurality of binary classifiers comprised in the classification module 120. Alternatively, the training sample can be used for training of the estimation model comprised in the estimation sub-module 142. By taking the second measurement value of the period information measured by the segmentation measurement module 140 as a training sample to train the corresponding model, it is possible to implicitly increase the supervision information for the corresponding model in the measurement apparatus 300, thereby improving the accuracy of the measurement of the period information and avoiding overfitting.

In some embodiments, the estimation sub-module 142 can use the information about feature points as a training sample to train the estimation model in the estimation sub-module 142. Therefore, in the case that the information about feature points is known, the information about feature points can be used as explicit supervision information to optimize the training of the model, thereby further improving the accuracy of the measurement of the period information and avoiding overfitting.

For example, the estimation sub-module 142 can further output the information about feature points based on the estimation results, as illustrated by the dotted arrow in FIG. 3. The information about feature points can be the amount of feature points in the corresponding segment. For example, the information about feature points can be used for training of various models in the measurement apparatus 300.

In some embodiments, training data can be used to jointly train one or more of the feature acquisition module 110, the classification module 120, the period information determination module 130, and the segmentation measurement module 140 in the measurement apparatus 300. That is, the feature acquisition module 110, the classification module 120, the period information determination module 130 and the segmentation measurement module 140 in the measurement apparatus 300 can be formed as an end-to-end learning model.

In some embodiments of the present disclosure, by using the segmentation measurement module 140, the accuracy of the measurement of the period information can be further improved and overfitting can be avoided.

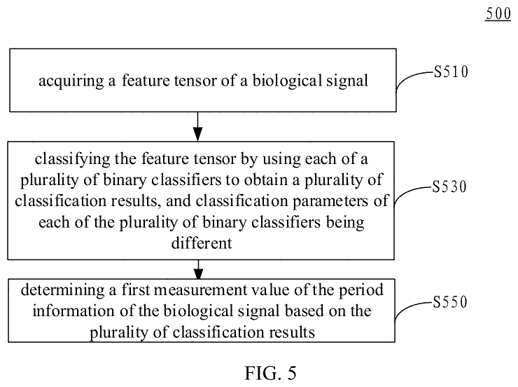

FIG. 5 illustrates a flowchart of the measurement method 500 for period information of a biological signal according to some embodiments of the present disclosure.

Referring to FIG. 5, in step S510, the feature tensor of the biological signal is acquired.

In the embodiment of the present disclosure, the biological signal comprises at least one selected from the group consisting of an electrocardiograph signal, a respiratory signal, a pulse signal, an electroencephalogram signal and an electromyography signal. The period information of the biological signal may comprise one or more of heart rate, respiratory rate, pulse rate, blink rate, etc.

In some embodiments, step S510 can comprise receiving a feature tensor of a biological signal from another device or module (for example, a memory).

In the embodiments of the present disclosure, the feature tensor may comprise one or more feature matrices for characterizing various features of the biological signal. In an example, features related to period information of a biological signal can be extracted to generate a third-order feature tensor. Elements in the third-order feature tensor are related to these features.

In the embodiments of the present disclosure, in the case that the biological signal is an electrocardiograph signal, the examples of the features related to the period information of the biological signal may comprise one or more of an R-wave peak (for example., an R-wave vertex), a T wave peak (for example, a T wave vertex), a QRS start point, or a QRS end point.

In some embodiments, step S510 may comprise extracting the features of the biological signal to obtain a feature tensor. For example, the features of the biological signal can be extracted by the first neural network model to obtain the feature tensor.

For example, the first neural network model may comprise a feedforward neural network, a recurrent neural network (RNN), a convolutional neural network (CNN), or other forms of neural networks. The type of the first neural network model may comprise a depth neural network.

Although it has been described that features of the biological signal can be extracted by various neural networks to obtain a feature tensor, the present disclosure is not limited thereto. For example, feature extraction can be performed on the biological signal through any machine learning model that can acquire the feature tensor.

In some embodiments, preprocessing the biological signal can be comprised before step S510.

For example, preprocessing the biological signal may comprise: filtering the biological signal to remove power frequency interference and/or baseline drift; and performing waveform detection and/or waveform segmentation on the biological signal that is filtered.

In the case of comprising preprocessing the biological signal, step S510 may comprise: after preprocessing the biological signal, performing feature extraction on the biological signal that is preprocessed to extract the feature tensor.

In some embodiments, because the scale or dimension of the biological signal may change, the dimension of the feature obtained by performing feature extraction on the biological signal may also change. That is, the order of the feature tensor that is obtained may vary depending on the scale or dimension of the biological signal.

Then, in step S530, the feature tensor is classified by using each of a plurality of binary classifiers to obtain a plurality of classification results, and the classification parameters of each of the plurality of binary classifiers are different.

In some embodiments, the inputs of the plurality of binary classifiers are the same. That is, the plurality of binary classifiers classify the same input. For example, the input of the plurality of binary classifiers may be the feature tensor obtained by performing feature extraction on the features of the biological signal. In an example, the input of the plurality of binary classifiers may be the feature vector obtained by converting the feature tensor. The example method described above can be adopted to convert the feature tensor into a feature matrix. Then, the feature matrix can be converted into a feature vector in the row direction or column direction of the feature matrix.

Next, in step S550, a first measurement value of the period information of the biological signal is determined based on the plurality of classification results.

In some embodiments, the first measurement value of the period information of the biological signal can be determined based on equation (1).

In some embodiments, before step S530, the measurement method may further comprise: determining the range of the period information of the biological signal; and determining the amount of the plurality of binary classifiers and the classification parameters of each of the plurality of binary classifiers based on the range.

In an example, in the case where the range of the period information is expressed as MIN˜MAX, and MIN and MAX are non-negative integers, the total amount of categories K can be expressed as: K=MAX-MIN+1, and category 1 to category K can correspond to MIN, MIN+1, . . . , Max, respectively. In this case, the amount (i.e., N) of the plurality of binary classifiers can be determined as the amount of categories K−1. Moreover, the classification parameters of the plurality of binary classifiers can be determined as MIN, MIN+1, . . . , MAX−1, respectively. By training each of the plurality of classifiers, the corresponding classifier can have corresponding classification parameters, and can have the ability to identify whether the inputs meet the classification condition related to the classification threshold. For example, after training, the binary classifier 1 can identify whether the input is greater than the classification threshold MIN, the binary classifier 2 can identify whether the input is greater than the classification threshold MIN+1, and the binary classifier N can identify whether the input is greater than the classification threshold MAX−1.

In some embodiments, each of the plurality of binary classifiers can be implemented as a neural network. For example, the neural network used for the classification module may comprise a feedforward neural network, a recurrent neural network (RNN), a convolutional neural network (CNN), or other forms of neural networks. The type of the neural network used for the classification module may comprise the depth neural network.

In some embodiments, the measurement method 500 may further comprise: training each of the plurality of binary classifiers.

For example, training each of the plurality of binary classifiers may comprise: determining a training data set; and training each of the plurality of binary classifiers by using the training data set.

For example, training each of the plurality of binary classifiers by using the training data set comprises: for each of the plurality of binary classifiers, dividing the training data set into a first subset and a second subset, and training the corresponding binary classifier by using the first subset and the second subset.

For training each of the plurality of binary classifiers, reference can be made to the previous description.

In some embodiments, the method 500 can be implemented by using the measurement apparatus 100 that is described with reference to FIG. 1.

In some embodiments of the present disclosure, the plurality of binary classifiers are trained in a multi-task manner based on different division. Because each binary classifier uses all the training samples in the training data set, it is not easy to be affected by local interference and is not easy to overfit.

The period information of the biological signal has significant incremental characteristic. For example, heart rate increases monotonically with the shortening of the heartbeat cycle. Moreover, the biological signal itself is easily disturbed by random noise, thereby resulting in randomness of its period information. In the embodiments of the present disclosure, the influence of random noise on the measurement results can be reduced by setting a plurality of binary classifiers for classification by using the incremental characteristic of the period information of the biological signal.

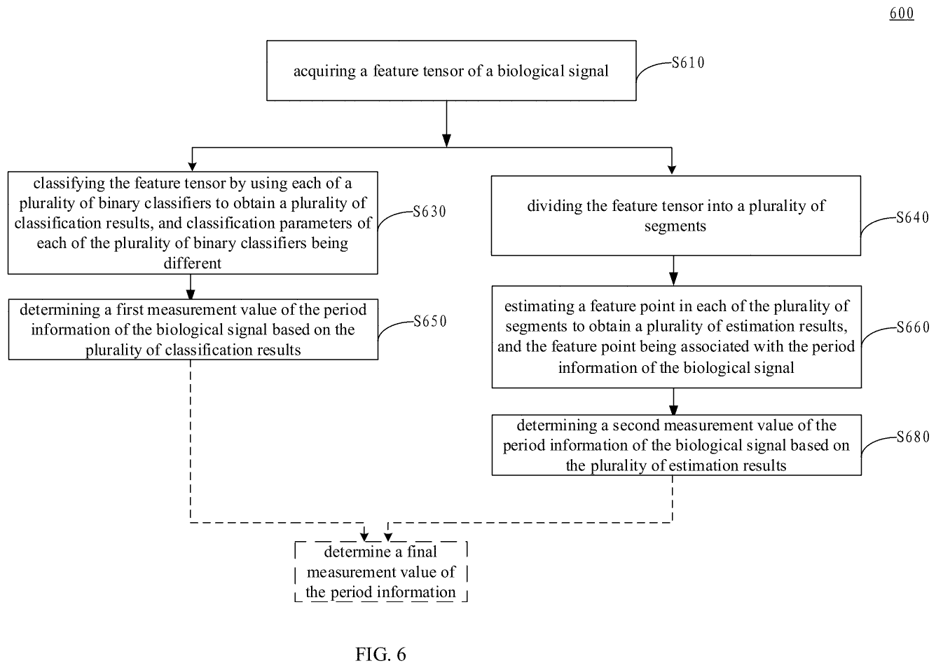

FIG. 6 illustrates a flowchart of a measurement method 600 for period information of a biological signal according to some embodiments of the present disclosure.

Referring to FIG. 6, in step S610, the features of the biological signal are extracted to obtain the feature tensor.

Then, in step S630, the feature tensor is classified by using each of the plurality of binary classifiers to obtain a plurality of classification results, and the classification parameters of each of the plurality of binary classifiers are different.

Next, in step S650, a first measurement value of the period information of the biological signal is determined based on the plurality of classification results.

Various embodiments of step S610, step S630 and step S650 can refer to step S510, step S530 and step S550, respectively, and detailed description thereof is omitted here.

In addition to step S610, the measurement method 600 further comprises step S640, step S660 and step S680. It should be noted that the embodiments of the present disclosure do not limit the order between step S640, step S660 and step S680, and step S630 and step S650. For example, step S640 can be performed before or after step S630, or in parallel with step S630.

Continuing to refer to FIG. 6, in step S640, the feature tensor is divided into a plurality of segments.

In some embodiments, step S640 may comprise converting the feature tensor into a feature matrix and dividing the feature matrix into a plurality of segments based on the column vectors of the feature matrix, and each of the plurality of segments corresponds to a column vector in the feature matrix.

In the embodiments of the present disclosure, the feature tensor can be converted into a feature matrix by using the method for converting the tensor into a matrix. For the method of converting the feature tensor into the feature matrix, reference can be made to various examples described above.

In some embodiments, in the case that the feature matrix is divided, it is ensured that the amount of feature points in each of the plurality of segments does not exceed one.

Next, in step S660, a feature point in each of the plurality of segments is estimated to obtain a plurality of estimation results, and the feature point is associated with the period information of the biological signal.

In some embodiments, in the case that the biological signal is an electrocardiograph signal, the feature point may comprise an R-wave peak (for example, an R-wave vertex). In this case, the period information of the biological signal can be measured by the amount of feature points of the same kind appearing in a certain period of time. For example, heart rate can be determined by the amount of R-wave vertices appearing in the electrocardiograph signal for a predetermined time period (for example, one minute).

In some embodiments, in the case that the feature matrix is divided, it is ensured that the amount of feature points in each of the plurality of segments does not exceed one. In this case, the period information of the biological signal can be obtained by estimating the amount of segments with feature points in the plurality of segments.

Then, in step S660, a second measurement value of the period information of the biological signal is determined based on the plurality of estimation results.

In some embodiments, the second measurement value of the period information of the biological signal can be determined based on equation (4) described above.

In some embodiments, the biological signal and the second measurement value of the period information corresponding to this biological signal can be taken as a training sample. For example, the training sample can be used for training the plurality of binary classifiers. By using the second measurement value of the period information that is measured as a training sample to train the corresponding model, it is possible to implicitly increase the supervision information for the corresponding model in the method 500, thereby improving the accuracy of the measurement of the period information, and avoiding overfitting.

In some embodiments, the information about feature points can be used as a training sample to train the estimation model for estimating the feature points in each of the plurality of segments. Therefore, in the case that the information about the feature points is known, the information about the feature points can be used as explicit supervision information to optimize the training of the model, thereby further improving the accuracy of the measurement of the period information and avoiding overfitting.

In some embodiments, as illustrated by the dotted box in FIG. 6, one of the first measurement value and the second measurement value can be taken as the final measurement value, and the final measurement value is taken as the period information of the biological signal that is obtained by measurement.

In some embodiments, the measurement method 600 may further comprise: obtaining the final measurement value of the period information of the biological signal based on the first measurement value and the second measurement value, and taking the final measurement value as the period information of the biological signal that is obtained by measurement.

For example, the first measurement value and the second measurement value can be weighted and averaged to obtain the final measurement value.

In some embodiments of the present disclosure, by using an additional segmentation measurement process, the accuracy of the measurement of period information can be further improved and overfitting can be avoided.

The measurement method for period information of a biological signal according to various embodiments of the present disclosure has been described above. It should be understood that the flowchart and block diagram in the attached drawings illustrate the architecture, functions and operations of possible implementations of the method, the apparatus, the system and the computer-readable storage medium according to various embodiments of the present disclosure. For example, each block in the flowchart or the block diagram can represent a module, a segment, or a code portion comprising at least one executable instruction for implementing a specified logical function. It should also be noted that in some alternative embodiments, the functions mentioned in the block may not occur in the order indicated in the attached drawings. For example, depending on the functions involved, two blocks shown in succession may actually be executed substantially simultaneously, or the blocks may sometimes be executed in a reverse order. It should also be noted that each block of the block diagram and/or flowchart and the combination of blocks in the block diagram and/or flowchart can be implemented by a dedicated hardware-based system that performs a specified function or action, or a combination of the dedicated hardware and computer instructions.

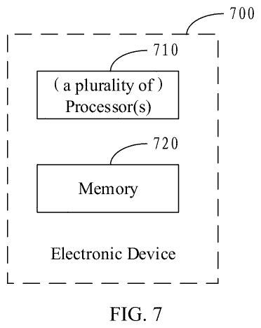

FIG. 7 illustrates a block diagram of an electronic device 700 according to some embodiments of the present disclosure.

Referring to FIG. 7, the electronic device 700 may comprise one or more processors 710 and a memory 720. The memory can be used to store one or more computer programs. The processor may comprise various processing circuits, such as, but not limited to one or more of a dedicated processor, a central processing unit, or an application processor. The memory may comprise a volatile memory and/or a non-volatile memory.

In some embodiments, in the case that one or more computer programs are executed by one or more processors, one or more processors are allowed to implement the method of the present disclosure described above.

In some embodiments, the electronic device 700 may comprise one or more sensors (not illustrated).

For example, one or more sensors can be used to sense a user's biological signal. For example, one or more sensors may comprise a photoplethysmography (PPG) sensor.

For example, one or more sensors can be provided as a part of the electronic device 700 or separately provided from the electronic device 700. In an example, one or more sensors can be provided on the surface of the electronic device 700.

For example, one or more processors 710 can execute one or more computer programs stored in the memory 720 to implement the method of the present disclosure described above, so as to measure the period information of the biological signal that is sensed by one or more sensors.

In some embodiments, the electronic device 700 in the embodiments of the present disclosure may comprise, for example, a smart phone, a tablet personal computer (PC), a server, a mobile phone, a video phone, an e-book reader, a desktop PC, a laptop computer, a netbook computer, a personal digital assistant (PDA), a portable multimedia player (PMP), an MP3 player, a mobile medical device, a camera or a wearable device (for example, a head mounted device (HMD), an electronic cloth, an electronic bracelet, an electronic necklace, an electronic jewelry, an electronic tattoo or a smart watch), etc.

As used herein, the term “module” can comprise units that are configured in hardware, software or firmware and/or any combination thereof and can be used interchangeably with other terms (for example, logic, logic block, component, or circuit). A module can be a single integral component or the smallest unit or component that performs one or more functions. The module can be implemented mechanically or electronically and can comprise, but is not limited to, dedicated processors, CPUs, application-specific integrated circuit (ASIC) chips, field programmable gate arrays (FPGA) or programmable logic devices that are known or to be developed to perform certain operations.

According to the embodiments of the present disclosure, at least a part of a device (for example, a module or a function thereof) or a method (for example, an operation or a step) can be implemented as an instruction stored in a computer-readable storage medium (for example, the memory 720) in the form of a program module, for example. When the instruction is executed by a processor (for example, the processor 710), the instruction can enable the processor to perform a corresponding function. The computer-readable storage medium can comprise, for example, a hard disk, a floppy disk, a magnetic medium, an optical recording medium, a DVD, a magneto-optical medium. The instruction can comprise codes created by the compiler or codes executable by the interpreter. The module or programming module according to various embodiments of the present disclosure can comprise at least one or more of the above components, some of which can be omitted, or other additional components can further be comprised. Operations performed by modules, programming modules, or other components according to various embodiments of the present disclosure can be performed sequentially, in parallel, repeatedly, or heuristically, or at least some operations can be performed or omitted in a different order, or other operations can be added.

For the present disclosure, the following points need to be explained.

(1) The attached drawings of the embodiments of the present disclosure only relate to the structures related to the embodiments of the present disclosure, and other structures can refer to the general design.

(2) In the case of no conflict, the embodiments of the present disclosure and the features in the embodiments can be combined with each other to obtain new embodiments.

What have been described above are only exemplary implementations of the present disclosure and are not used to limit the protection scope of the present disclosure, and the protection scope of the present disclosure is determined by the appended claims.

1. A measurement method for period information of a biological signal, comprising:
acquiring a feature tensor of the biological signal;
classifying the feature tensor by using each of a plurality of binary classifiers to obtain a plurality of classification results, and classification parameters of each of the plurality of binary classifiers being different; and
determining a first measurement value of the period information of the biological signal based on the plurality of classification results.

2. The measurement method according to claim 1, before classifying the feature tensor by using each of the plurality of binary classifiers, further comprising:
determining a range of the period information of the biological signal; and
determining an amount of the plurality of binary classifiers and the classification parameters of each of the plurality of binary classifiers based on the range.

3. The measurement method according to claim 1, wherein
classifying the feature tensor by using each of the plurality of binary classifiers to obtain the plurality of classification results comprises:
converting the feature tensor into a feature matrix; and
classifying the feature matrix by using each of the plurality of binary classifiers to obtain the plurality of classification results.

4. The measurement method according to claim 1, wherein each of the plurality of binary classifiers is implemented as a convolutional neural network, and
acquiring the feature tensor of the biological signal comprises extracting features of the biological signal through the convolutional neural network so as to acquire the feature tensor.

5. The measurement method according to claim 1, further comprising:
dividing the feature tensor into a plurality of segments;
estimating a feature point in each of the plurality of segments to obtain a plurality of estimation results, and the feature point being associated with the period information of the biological signal;
determining a second measurement value of the period information of the biological signal based on the plurality of estimation results; and
obtaining a final measurement value of the period information of the biological signal based on the first measurement value and the second measurement value.

6. The measurement method according to claim 1, further comprising:
dividing the feature tensor into a plurality of segments;
estimating a feature point in each of the plurality of segments to obtain a plurality of estimation results, and the feature point being associated with the period information of the biological signal; and
determining a second measurement value of the period information of the biological signal based on the plurality of estimation results,
wherein the biological signal and the second measurement value further serve as training data for training of each of the plurality of binary classifiers.

7. The measurement method according to claim 5, wherein dividing the feature tensor into the plurality of segments comprises:
converting the feature tensor into a feature matrix; and
dividing the feature matrix into the plurality of segments.

8. The measurement method according to claim 5, wherein a convolutional neural network is used to estimate the feature point in each of the plurality of segments, and
the plurality of estimation results are further used for training of the convolutional neural network.

9. The measurement method according to claim 1, further comprising: training each of the plurality of binary classifiers,
wherein the training comprises:
determining a training data set for the plurality of binary classifiers;
dividing the training data set into a first subset and a second subset for each of the plurality of binary classifiers; and
training a corresponding binary classifier by using the first subset and the second subset.

10. The measurement method according to claim 1, wherein a type of the biological signal comprises at least one selected from a group consisting of an electrocardiograph signal, a respiratory signal, a pulse signal, an electroencephalogram signal, and an electromyography signal.

11. A measurement apparatus for period information of a biological signal, comprising:
a feature acquisition module, configured to acquire a feature tensor of the biological signal;
a classification module, configured to classify the feature tensor by using each of a plurality of binary classifiers to obtain a plurality of classification results, classification parameters of each of the plurality of binary classifiers being different; and
a period information determination module, configured to determine a first measurement value of the period information of the biological signal based on the plurality of classification results.

12. An electronic device, comprising:
at least one processor; and
a memory, configured to store at least one computer program,
wherein, in a case where the at least one computer program is executed by the at least one processor, the at least one processor executes the measurement method according to claim 1.

13. The electronic device according to claim 12, further comprising one or more sensors for acquiring the biological signal to be measured.

14. A computer-readable storage medium, on which at least one computer program is stored, wherein, in a case where the at least one computer program is executed by at least one processor, the at least one computer program executes the measurement method according to claim 1.

15. The measurement method according to claim 2, wherein classifying the feature tensor by using each of the plurality of binary classifiers to obtain the plurality of classification results comprises:
converting the feature tensor into a feature matrix; and
classifying the feature matrix by using each of the plurality of binary classifiers to obtain the plurality of classification results.

16. The measurement method according to claim 2, wherein each of the plurality of binary classifiers is implemented as a convolutional neural network, and
acquiring the feature tensor of the biological signal comprises extracting features of the biological signal through the convolutional neural network so as to acquire the feature tensor.

17. The measurement method according to claim 3, wherein each of the plurality of binary classifiers is implemented as a convolutional neural network, and
acquiring the feature tensor of the biological signal comprises extracting features of the biological signal through the convolutional neural network so as to acquire the feature tensor.

18. The measurement method according to claim 6, wherein dividing the feature tensor into the plurality of segments comprises:
converting the feature tensor into a feature matrix; and
dividing the feature matrix into the plurality of segments.

19. The measurement method according to claim 18, wherein a convolutional neural network is used to estimate the feature point in each of the plurality of segments, and
the plurality of estimation results are further used for training of the convolutional neural network.

20. The measurement method according to claim 2, further comprising:
dividing the feature tensor into a plurality of segments;
estimating a feature point in each of the plurality of segments to obtain a plurality of estimation results, and the feature point being associated with the period information of the biological signal;
determining a second measurement value of the period information of the biological signal based on the plurality of estimation results; and
obtaining a final measurement value of the period information of the biological signal based on the first measurement value and the second measurement value.
